(19) United States
(12) Patent Application Publication
(10) Pub. No.: US 2008/0095856 A1
(43) Pub. Date: Apr. 24, 2008

(54) Encapsulated Nanoparticles for Drug Delivery

(76) Inventors: Gunilla B. Jacobson, Menlo Park, CA (US); Richard N. Zare, Stanford, CA (US); Karin E. Markides, Provo, UT (US); Rajesh R. Shinde, Stanford, CA (US); Christopher H. Contag, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/748,408

(22) Filed: May 14, 2007

Related U.S. Application Data
(60) Provisional application No. 60/800,137, filed on May 12, 2006.

(51) Int. Cl. A61K 9/14; A61K 31/70; A61K 38/02

(52) U.S. Cl. 424/497; 514/2; 514/44; 977/773

(57) ABSTRACT

Compositions and methods are provided for preparing nanosized biologically active agents, including agents formulated for target specific drug delivery. The nanosized agents are prepared with supercritical carbon dioxide as an antisolvent, providing nanoparticles whose size, shape, and surroundings are well-controlled. The nanoparticles are made of small molecules, e.g. drugs, anti-oxidants, luciferin, polypeptides, e.g. oligopeptides; polynucleotides, e.g. siRNA, antisense oligonucleotides, etc. In some embodiments, the nanoparticles comprise a polymer coating, which can provide for controlled delivery, targeting, controlled release, and the like. In other embodiments, the nanoparticles comprise a target specific tag for targeting the nanoparticles to a site of interest, e.g. tissue, cell, etc.

### CROSS-REFERENCE TO RELATED APPLICATIONS

This applications claims benefit of priority to U.S. provisional application 60/800,137, filed May 12, 2006, which is herein incorporated by reference.

### FIELD OF THE INVENTION

The present invention relates generally to drug delivery, including target specific drug delivery, and delivery of nucleic acids.

### BACKGROUND OF THE INVENTION

During the last five years nanotechnology has had a significant impact on several technologies, such as electronics, mechanical structures, catalysis, and processing. However, the use of nanotechnology in biomedical applications has just started, including areas such as target-specific drugs, nanostructured biomaterial for biointegration, nanoprobes for cellular targeting, nanofluidic chips for DNA processing, and drug delivery. Nanotechnology has the potential to link the structure and function of biomolecules to the actual physiological event and allow for a more detailed understanding of biological systems.

The definition of nanotechnology involves the use of materials with a length scale less than 100 nm in at least one dimension. This dimension is a perfect fit with the size of biological structures that range from the tens of nanometers (proteins, DNA, viruses) to hundreds of nanometers (cells and cellular assemblies). Controlled nanosize is the key to why nanoparticles will have a significant impact on drug delivery and target-specific pharmaceuticals.

Currently used methods to transport nanoparticles of pharmaceuticals include liposomes, carbon nanotubes, micelles, polymeric nanoparticles, etc. Desirable properties of these carriers include increased longevity in the blood and thereby accumulation in the pathological area, targeted specific delivery; increased intracellular penetration, controlled release, e.g. by heat or pH changes; and in vivo imaging, e.g. by contrast moieties.

Many of the current efforts for nanoscale manufacturing are targeting the “bottom-up” approach, where single molecules are assembled together in a specific pattern. Techniques used include scanning probe instruments, nanoscale lithography, and self-assembly techniques (see, for example, Torchilin Nat. Rev. Drug Discovery 2005, 4, 145-160; Lopez-Quintela, et al. Curr. Opin. Colloid. Interface Sci. 2004, 9. 264-278). Alternatively, a “top-down” process may be used. Current methods using the “top-down” approach utilizes lithography and requires processes such as ion etching, baking, ultrasonication, and solvent processing (see Xia et al. Chem. Rev. 1999, 99, 1823-1848). However, these processes are compatible with inorganic material, but are too harsh for organic, and especially bioactive, compounds.

For organic materials, currently used methods for particle formation include crystallization and precipitation, for example using liquid antisolvents or emulsions. This processes have a disadvantage of high heat requirements, organic solvent residues, large (micron-sized) and non-uniform particles size, as well as loss of yield due to several precipitation/purifications steps. To further reduce the particle size, techniques such as grinding, milling, and crushing can be used, but are not always compatible with biologically active compounds due to thermal and chemical degradation and well as shock sensitivity.

### SUMMARY OF THE INVENTION

Compositions and methods are provided of nanosized biologically active agents, including agents formulated for target specific drug delivery. The nanosized agents are prepared with supercritical carbon dioxide as an antisolvent, providing nanoparticles whose size, shape, and surroundings are well-controlled. The nanoparticles are made of small molecules, e.g. drugs, anti-oxidants, luciferin, polypeptides, e.g. oligopeptides; polynucleotides, e.g. siRNA, antisense oligonucleotides, etc. In some embodiments, the nanoparticles comprise a polymer coating, which can provide for controlled delivery, targeting, controlled release, and the like. In other embodiments, the nanoparticles comprise a target specific tag for targeting the nanoparticles to a site of interest, e.g. tissue, cell, etc.

In one embodiment of the invention, methods are provided for the preparation on biologically active agents in nanoparticle form. The process utilizes supercritical carbon dioxide (SC-CO2) as an antisolvent for rapid and controlled precipitations. No purification or drying steps are needed, and the process is compatible with bioactive compounds, including drugs, peptides, proteins, nucleotides, polynucleotides, and the like. The method is easily scaled up to high volume manufacturing.

### BRIEF DESCRIPTION OF THE DRAWINGS

FIG. 1. Supercritical Antisolvent System.

FIGS. 2A-2B. SEM of Luciferin 2A: before and 2B: after SAS processing.

FIGS. 3A-3C. SEM of Quercetin 3A: before SAS, and after SCF processing using 3B: methanol, and 3C: isopropanol as cosolvents.

FIGS. 4A-4C: After SAS processing. A: quercetin, B: PLA and C: quercetin/PLA.

FIGS. 5A-5C. After SAS processing. A: luciferin, B: luciferin/chitosan, and C: Luciferin/PLGA.

FIGS. 6A-6F. Effects of parameters on encapsulation. All three SEM pictures on each row are the same structures shown at different magnifications. Parameters are those set forth in Table 1. (A) is PLA in the absence of luciferase. (B) 9% luciferase, PLA 100K. (C) 3% luciferase, PLA 50K, in DMSO. (D) 10% luciferase, PLA 50K, in MeOH; (E) 1% luciferase, PLA 50K; (F) 4% luciferase, PLA 50K.

FIGS. 7A-7B. SEM of tRNA before (A) and after SAS (B).

FIGS. 8A-8B. SEM of PLA (MW 100,000) encapsulated tRNA, in a 5:100 wt % ratio of tRNA to PLA.

FIGS. 9A-9B. SEM of PLA (MW 50,000) encapsulated tRNA, in a 5:100 wt % ratio of tRNA to PLA.

FIGS. 10A-10B. SEM of siRNA after SAS.

FIGS. 11A-11B. A: HPLC analysis of encapsulated siRNA, and B: gel electrophoresis of encapsulated siRNA using 15% PAGE.

FIG. 12. SEM of siRNA encapsulated with PLA (MW=100,000) (Both images from the same sample)

FIG. 13. Silencing assay of siRNA before and after SAS process.

### DETAILED DESCRIPTION OF THE EMBODIMENTS

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Compositions and methods are provided of nanosized biologically active agents, including agents formulated for target specific drug delivery. The nanosized agents are prepared with supercritical carbon dioxide as an antisolvent, providing nanoparticles whose size, shape, and surroundings are well-controlled. The nanoparticles are made of small molecules, e.g. drugs, siRNA, antisense oligonucleotides, etc. In some embodiments, the nanoparticles comprise a polymer coating, which can provide for controlled delivery and release. In other embodiments, the nanoparticles comprise a target specific tag for targeting the nanoparticles to a site of interest, e.g. tissue, cell, etc.

When a fluid is taken above its critical temperature (Tc) and critical pressure (Pc), it exists in a condition called a supercritical fluid (SCF) state in which it is no longer described exclusively as a gas or a liquid. The unique characteristics of a SCF is that one can control the physical properties of the fluid, e.g. density, viscosity, diffusivity, by small changes in temperature or pressure (see Jacobson et al. J. Am. Chem. Soc.; 1999; 121(50); 11902-11903). Carbon dioxide is the most popular SCF because of its physiological compatibility, non-toxicity, low critical parameters (Tc=31° C., Pc=7.38 MPa), inexpensiveness, and relative environmental friendliness (see Ikariya et al. Green Chemistry Using Liquid and Supercritical Carbon Dioxide, Ed. J. DeSimone, 2000).

SCFs are used in the preparation of nanoparticles whose size and surroundings can be controlled for the use in pharmaceutical and imaging applications. The starting material containing the compound of interest; or the compound of interest and the polymer for encapsulation, is dissolved in a suitable cosolvent, which cosolvent mixture is then sprayed into a fluid, usually a supercritical CO₂ fluid acting as an antisolvent, that is, a supercritical fluid in which the compound of interest has limited solubility (see Chattopadhyay et al. AlChE J. 2002, 48, 235-244). In some embodiments, however, the antisolvent of interest may be at a subcritical state, e.g. at a pressure where CO₂ is supercritical at 32° C., the fluid may be at a slightly lower temperature, e.g. around about 25° C., around about 28° C., around about 30° C.

As the solvent is expanded into the supercritical solution, rapid precipitation of the target compound is achieved, allowing for dry particles to be collected after depressurization and venting of the SCF/cosolvent mixture. The particle size and particle size distribution is controlled by changes in temperature, pressure, flow rate, cosolvent, and concentration of the target compound, for example as described by Reverchon et al. Int J. Pharm., 2002, 243, 83-91.

The use of a SCF as the antisolvent improves many of the drawbacks of liquid antisolvents (see Wang et al. AlChE J, 2005, 51, 440-455). The antisolvent is completely removed by pressure reduction, eliminating the need for additional post-treatment steps. Also, the high diffusivity of SCFs allows much faster diffusion into the liquid solvent and formation of supersaturation of the solute. This, in turn, allows for much smaller nanosized particles to be formed as well as control of the size distribution, as compared to using liquid antisolvents, or other techniques such as jet milling. Consequently, this technique has the advantage of allowing significant scale-up for the production of large quantities of nanoparticles (kilogram amounts).

The methods of the invention also provide for encapsulation of the nanoparticles in situ in the SAS process. Encapsulating bioactive compounds may provide one or more of: protection of the active compound in the core of the particle against heat and oxygen; retention of nano-properties that can be lost by cluster formation; enhanced targeting to a site of interest; and controlled-release of the compound. It has for instance been shown that an outer polymer-layer protects volatile compounds from evaporation (see Mathis et al., Journal of Medicinal Chemistry, 2003, 46, 274). Encapsulation can modify the surface of the nanoparticle for various purposes, such as target specificity, time release, and controlled biodistribution. A thin coating of a polymer, surfactant, or target-specific tag, encapsulates the particle to change its surface properties. Engineering of specific properties such as flowability, dissolution rate, dispersability, chemical reactivity, bioefficacy, and hydrophilicity are available for a range of applications. (see Davies et al. Adv. Mater. 1998, 10, 1264-1270; Wang et al. J. Controlled Release, 1999, 57, 9-18).

Conventional methods for encapsulation of fine particles include emulsion evaporation, phase separation, spray-drying, and freeze-drying. The common disadvantages of these techniques are that they require large amounts of organic solvents, surfactants, and additives, as well as yielding low encapsulation efficiency. They also require further downstream processing, such as drying, milling, and sieving. As a result, residual toxic solvents, extreme temperature and pH requirements, and strong shear forces can all affect adversely nanosized biomaterials. The methods of the present invention address these issues.

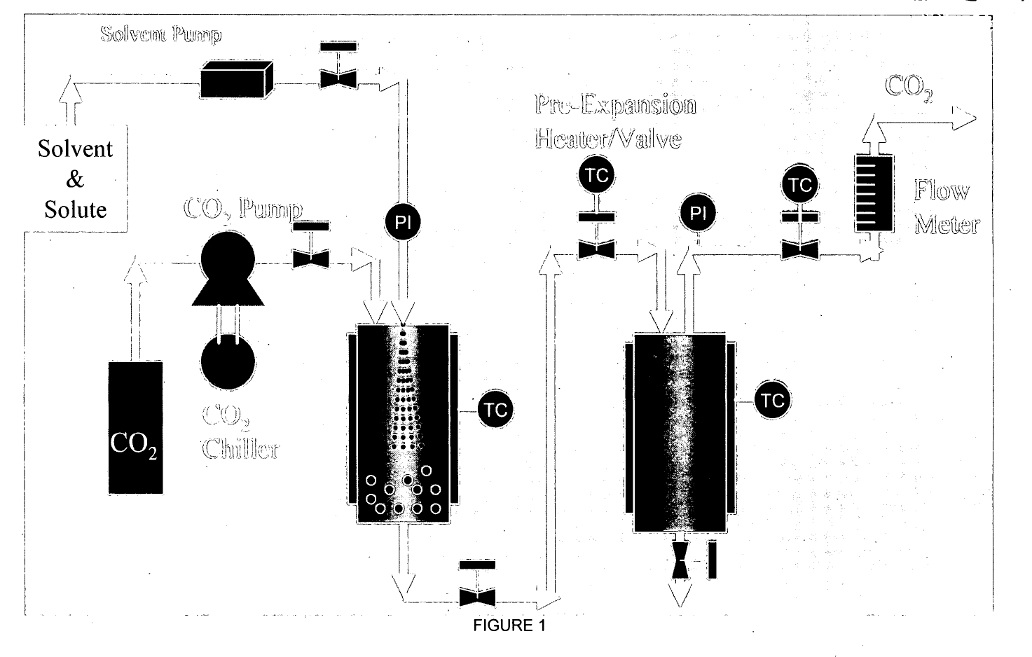

The supercritical antisolvent process of the invention is shown in FIG. 1. The active agent is solubilized in a cosolvent. This cosolvent typically contains a purified composition of the active agent, or the active agent and polymer being used for encapsulation. The cosolvent may be a single molecular entity, e.g. water, methanol, ethanol, acetone, isopropanol, dimethyl sulfoxide, dimethyl formamide, methylene chloride, chloroform, ethyl acetate, tetrahydrofuran, toluene, N-methylpyrrolidone, etc. The cosolvent is chosen to be an entity in which the active agent is soluble.

In some embodiments, the active agent and the polymer are soluble in a single entity, as described above. In other embodiments, the active agent and the polymer are not soluble in the same molecular entity. In such cases each will be separately dissolved into entities that are completely miscible with each other, and which are combined to provide a homogeneous mixture, either before or after dissolving the polymer and active agent. In such embodiments, the term cosolvent may refer to such a homogenous mixture.

For example, in the methods utilizing luciferin and PLA, luciferin is dissolved in methanol and PLA in dichloromethane. The methanol and dichloromethane solutions are completely miscible.

The concentration of the active agent and the polymer in the cosolvent, as well as the active agent/polymer ratio, allows for control of particle size and encapsulation yield. The concentrations are selected to provide for the desired end product by optimization, as is known in the art. In general, a lower concentration of active agent is selected for smaller particle sizes, and a higher concentration for larger particle sizes. A higher ratio of polymer to active agent will provide for a thicker polymer encapsulation, while a lower ratio of polymer to active agent will provide for a thinner coating. The concentration of active agent will usually be at least about 0.001 mg/ml, more usually at least about 0.01 mg/ml, at least about 0.1 mg/ml, or 1 mg/ml., and not more than about 100 mg/ml, usually not more than about 10 mg/ml. The concentration of polymer will usually be at least about 0.01 mg/ml, more usually at least about 0.1 mg/ml, at least about 1 mg/ml, and not more than about 100 mg/ml, usually not more than about 50 mg/ml. The ratio of compound to polymer as a weight percent will vary, from around about 1:1000; 1:500; 1:100, 1:50; 1:10; 1:5, and the like.

The co-solvent solution with active agent and polymer is sprayed at a set flow rate into a particle vessel filled with a continuous flow of supercritical carbon dioxide. The flow rate is usually a constant rate; however a drying step of CO₂ at varying rates may be performed in some embodiments. The flow rate of CO₂ is usually at least about 1 g/min of CO₂, more usually at least about 10 g/min of CO₂, at least about 50 g/min of CO₂, and not more than about 1000 g/min of CO₂, not more than about 500 g/min of CO₂, and may be around about 100 g/min of CO₂, around about 150 g/min of CO₂, or around about 200 g/min of CO₂. Flow rate may be optimized for each active agent/polymer/cosolvent system. The selection is based on the desired yield and particle size. In general, although with some exceptions, higher yields are achieved with lower flow rates, and smaller particles with a higher flow rate.

The volume of cosolvent solution injected into the vessel is determined, in part, by the scale of the reaction. In a laboratory scale reaction, e.g. having a vessel of around about 500 ml, the injected volume may be at least about 0.1 ml, at least about 0.5 ml., at least about 1 ml., and not more than about 100 ml, usually not more than about 50 ml, or not more than about 10 ml. It will be understood by one of skill in the art that a larger volume is appropriate for a manufacturing scale. The injection flow rate controls the concentration of cosolvent as well as of the compound to be precipitated. Lower flow rates are desired to keep the lowest concentrations, which allow for smaller particle sizes. The flow rate is usually at least about 0.01 ml/minute, at least about 0.1 ml/minute, at least about 0.5 ml/minute, at least about 5 ml/minute, and not more than about 100 ml/minute, usually not more than about 50 ml/minute, and may be less than about 10 ml/minutes.

As the cosolvent is expanded into the particle vessel, the cosolvent is immediately solubilized in the supercritical fluid, and the solute will instantly precipitate out of solution. The process conditions are selected so that the active agent is insoluble, and the cosolvent is completely soluble. The parameters of pressure and temperature at which the conditions are met may be determined by reviewing a phase diagram of the selected cosolvent and antisolvent, as known and available in the art.

Phase diagrams of particular interest include a cosolvent/CO₂ pair at the desired concentration of active agent and polymer. Some phase diagrams can be found in the literature, others are determined experimentally. Each phase diagrams shows the cloud point data as a function of temperature and pressure, at a specific concentration. The process conditions are selected to be above the cloud point, where pressure and temperature may each be varied.

Generally a temperature selected to maintain the stability of the active agent polymer, and is usually not more than about 100° C., more usually not more than about 80° C., and may be not more than about 40° C., 30° C., or 20° C. When a polymer is included it is desirable to keep the temperature below the glass transition temperature of the polymer, which typically ranges from 45-65° C., e.g. for PLGA. Therefore in some embodiments a temperature of around about 40° C. Is used to advantage. In some embodiments, the temperature is kept constant, and pressure is varied.

Pressure is used to tailor the solubility of cosolvent vs CO₂, and has a smaller effect on particle size. When a polymer is included a lower pressure is desired as increased pressures will decrease the glass transition temperature of the polymers. Generally a pressure is selected of at least about 50 bar, usually at least about 80 bar, and not more than about 1000 bar, usually not more than about 500 bar, and may be in the range of about 80 to 120 bar.

The particles are collected, e.g. on a filter at the bottom on the particle chamber. The supercritical fluid, which is now a mixture of carbon dioxide and cosolvent) is optionally further expanded into a coalescer at a lower pressure, causing the cosolvent to drop out of solution for further collection, and the carbon dioxide is now a gas which can be recycled and reused.

A benefit of the present invention is the ability to generate nanoparticles of controlled size and composition, where the size of particles in a population can be substantially homogeneous. The nanoparticles of the present invention comprise a solid core that is substantially pure active agent or drug, usually at least about 75% pure, at least 85% pure, at least about 95% pure, at least about 99% pure. It will be understood by one of skill in the art that two or more active compounds can be co-formulated, in which case the purity shall refer to the combined active agents.

The core of the nanoparticles has a controlled size. Usually the core is at least about 10 nm in diameter, more usually at least about 35 nm in diameter, at least about 50 nm in diameter. The core of the nanoparticles is usually not more than about 5 μm in diameter, not more than about 1 μm in diameter, and may be not more than about 500 nm in diameter, or not more than about 100 nm in diameter. Nanoparticles of nucleic acids, particularly oligonucleotides of less than about 200 nt in length may be small, e.g. of from about 10 nm to about 100 nm in diameter. The nanoparticles core may have a defined size range, which may be substantially homogeneous, where the variability may be not more than 100% of the diameter, not more 50%, not more than 10%, etc.

The nanoparticle core may be covered with a substantially uniform coating, where the coating may be any biologically compatible polymer. Some examples of biodegradable polymers useful in the present invention include hydroxyaliphatic carboxylic acids, either homo- or copolymers, such as poly(lactic acid), poly(glycolic acid), Poly(dl-lactide/glycolide, poly(ethylene glycol); polysaccharides, e.g. lectins, glycosaminoglycans, e.g. chitosan; celluloses, acrylate polymers, and the like. The selection of coating may be determined by the desired rate of degradation after administration, by targeting to a desired tissue, e.g. in the use of lectins, by protection from oxidation, and the like. The coated particle will typically have a size of at least about 10 nm in diameter, at least about 50 nm in diameter, at least about 100 nm in diameter, at least about 250 nm in diameter, and not more than 10 μm in diameter, not more than about 5 μm in diameter, or not more than about 1 μm in diameter.

The term “biologically active agent” as used herein describes any molecule, e.g. nucleic acid, polypeptide, pharmaceutical, etc. with a desired biological activity and suitable solubility profile. The methods of the invention find particular use with active agents, e.g. nucleic acids, that have a short half-life in vivo due to degradation.

Active agents of interest for the SAS process of the invention include, without limitation, pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Active agents encompass numerous chemical classes, though typically they are organic molecules, and may be biopolymers such as polypeptides and polynucleotides, or small organic compounds having a molecular weight of more than 50 and less than about 2,500 daltons. Agents are also found among biomolecules including peptides, saccharides, fatty acids, lipids, steroids, purines, pyrimidines, derivatives, structural analogs or combinations thereof.

Included in the active agents are genetic agents. As used herein, the term “genetic agent” refers to polynucleotides and analogs thereof. Genetic agents such as DNA can result in an introduced change in the genetic composition of a cell, e.g. through the integration of the sequence into a chromosome. Genetic agents such as antisense or siRNA oligonucleotides can also affect the expression of proteins without changing the cell's genotype, by interfering with the transcription or translation of mRNA. The effect of a genetic agent is to increase or decrease expression of one or more gene products in the cell.

A large number of public resources are available as a source of genetic sequences, e.g. for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746. cDNA clones corresponding to many human gene sequences are available from the IMAGE consortium. The international IMAGE Consortium laboratories develop and array cDNA clones for worldwide use. The clones are commercially available, for example from Genome Systems, Inc., St. Louis, Mo. Methods for cloning sequences by PCR based on DNA sequence information are also known in the art.

In one embodiment, the genetic agent is an antisense or siRNA sequence that acts to reduce expression of the targeted sequence. Antisense or siRNA nucleic acids are designed to specifically bind to RNA, resulting in the formation of RNA-DNA or RNA-RNA hybrids, with an arrest of DNA replication, reverse transcription or messenger RNA translation. Gene expression is reduced through various mechanisms. Antisense nucleic acids based on a selected nucleic acid sequence can interfere with expression of the corresponding gene.

In some embodiments, the nucleic acid is treated prior to the SAS process to increase the hydrophobicity of the active agent, e.g. by treatment with a cationic lipid, e.g. 1,2-Dioleoyl-3-Trimethylammonium-Propane (Chloride Salt) (DOTAP), prior to solubilizing with the polymer. For example the BLIGH DYER technique may be used for making the nucleic acid hydrophobic.

Antisense oligonucleotides (ODN), include synthetic ODN having chemical modifications from native nucleic acids, or nucleic acid constructs that express such anti-sense molecules as RNA. One or a combination of antisense molecules may be administered, where a combination may comprise multiple different sequences. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like.

Among nucleic acid oligonucleotides are included phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Nucleic acid molecules of interest also include nucleic acid conjugates. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed.

Also of interest are RNAi agents. RNAi agents are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

The coated nanoparticles may further comprise a targeting moiety, which may be covalently or non-covalently bound to the particle after formation and encapsulation. Alternatively, the coating itself may serve a targeting role, e.g. in the encapsulation with lectins, and the like.

A targeting moiety, as used herein, refers to all molecules capable of specifically binding to a particular target molecule and forming a bound complex. Thus the ligand and its corresponding target molecule form a specific binding pair.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

Examples of targeting moieties include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting moieties of particular interest include peptidomimetics, peptides, antibodies and antibody fragments (e.g. the Fab′ fragment). For example, β-D-lactose has been attached on the surface to target the aloglysoprotein (ASG) found in liver cells which are in contact with the circulating blood pool.

Cellular targets include tissue specific cell surface molecules, for targeting to specific sites of interest, e.g. neural cells, liver cells, bone marrow cells, kidney cells, pancreatic cells, muscle cells, and the like. For example, nanoparticles targeted to hematopoietic stem cells may comprise targeting moieties specific for CD34, ligands for c-kit, etc. Nanoparticles targeted to lymphocytic cells may comprise targeting moieties specific for a variety of well known and characterized markers, e.g. B220, Thy-1, and the like.

Endothelial cells are a target of particular interest, in particular endothelial cells found in blood vessels, e.g. during angiogenesis, inflammatory processes, and the like. Among the markers present on endothelial cells are integrins, of which a number of different subtypes have been characterized. Integrins can be specific for endothelial cells involved in particular physiological processes, for example certain integrins are associated with inflammation and leukocyte trafficking (see Alon & Feigelson (2002) Semin Immunol. 14(2):93-104; and Johnston & Butcher (2002) Semin Immunol 14(2):83-92, herein incorporated by reference). Targeting moieties specific for molecules such as ICAM-1, VCAM-1, etc. may be used to target vessels in inflamed tissues.

Endothelial cells involved in angiogenesis may be targeted for site directed delivery of nucleic acids. Diseases with a strong angiogenesis component include tumors growth, particularly solid tumor growth, psoriasis, macular degeneration, rheumatoid arthritis, osteoporosis, and the like. A marker of particular interest for angiogenic endothelial cells is the αvβ3 integrin. Ligands for this integrin are described, for example, in U.S. Pat. No. 5,561,148; No. 5,776,973; and No. 6,204,280; and in International patent publications WO 00/63178; WO 01/10841; WO 01/14337; and WO 97/45137, herein incorporated by reference.

### Pharmaceutical Compositions

The nanoparticles of the invention may be incorporated in a pharmaceutical formulation. Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The compositions of the invention may be administered using any medically appropriate procedure, e.g., intravascular (intravenous, intraarterial, intracapillary) administration. The effective amount of a therapeutic composition to be given to a particular patient will depend on a variety of factors, several of which will be different from patient to patient. A competent clinician will be able to determine an effective amount of a therapeutic agent. The compositions can be administered to the subject in a series of more than one administration. For therapeutic compositions, regular periodic administration (e.g., every 2-3 days) will sometimes be required, or may be desirable to reduce toxicity. For therapeutic compositions that will be utilized in repeated-dose regimens, antibody moieties that do not provoke immune responses are preferred.

Those of skill will readily appreciate that dose levels can vary as a function of the specific compound, the severity of the symptoms and the susceptibility of the subject to side effects. Some of the specific complexes are more potent than others. Preferred dosages for a given agent are readily determinable by those of skill in the art by a variety of means. A preferred means is to measure the physiological potency of a given compound.

### Experimental

The first model compound developed for the particle formation process (SAS process) was luciferin. The SCF process is applicable and scaleable to other target compounds as well, which has been determined using quercetin, tRNA and siRNA. An advantage of working with luciferin as a test compound is the ability to obtain rapid in vivo data by measuring bioluminescence, which allows evaluation of the effect of different parameters of both the process as well as the particle composition. Some of these parameters include particle size, particle size distribution, encapsulation procedure, polymer, and specific “tags”. Luciferin is an amphiphatic molecule with excellent biodistribution. Obtaining real-time in vivo readouts due to the localization and dissolution of the luciferin-encapsulated nanoparticles provided a unique platform to visualize and optimize the development of nano-drug delivery systems. An understanding of the polymer-coated luciferin nanoparticles allowed utilization of this process for other compounds.

Quercetin. Red wine contains valuable antioxidants such as a wide range of different polyphenols. A moderate consumption of red wine has been linked to lower risk of developing coronary heart disease. Several recent studies have shown that different types of natural polyphenols may have neuroprotective effects both in vitro and in vivo, possibly due to their electron scavenging properties. One of the potent compounds in red wine responsible for its antioxidative properties is quercetin. This molecule has mostly been studied for its electron scavenging activities in terms of reducing risks of cancer development. However, it has also been shown in a few studies that quercetin may prevent or slow down the development of Alzheimer disease. Several mechanisms for its mode of action have been proposed, e.g. by protecting hippocampal cells against toxic effects induced by Aβ-peptides, by inhibiting the activity of the cyclin-dependent kinase (Cdk5/p35) system, by hindering the polymerization of free Aβ-peptide into Aβ fibrils, and by increased gene expression for transthyretin, a protein preventing aggregation of Aβ-peptide.

Quercetin represents molecules with properties that differ from luciferin, and one for which there are readily available assays to assess the extent of encapsulation. Quercetin is difficult to solubilize in regular solvents or buffers. Methanol is the common solvent used to dissolve quercetin and is toxic for clinical applications. By generating polymer-coated nanoparticles, quercetin can be delivered appropriately to the target as the hydrophilic polymer coating will enhance its in vivo biodistribution. Quercetin is a model drug that presents challenges typically found in numerous other drugs, wherein the solubility and pharmacokinetic/pharmacodynamic characteristics make use difficult, and as such serves as another powerful model compound for validating the methods of the invention. Anti cancer drugs such as Taxol are administered using DMSO as the solvent due to its limited solubility in water. Use of nanoparticles with a hydrophilic surface provides a means to overcome these challenges.

Imaging of Drug Delivery. A reporter transgenic animal was used as a source of labeled cells for cell trafficking studies and for the study of luciferin biodistribution. This allows development of imaging substrates with enhanced function for use in animal models of human biology and disease. This animal model was adapted for the development of improved tools that more effectively deliver small molecules to cellular targets in the living body. In this way luciferin served as a model small molecule, that after conversion into coated nanoparticles allows real-time biodistribution readout in the transgenic animal. Many cell types (e.g., lymphocytes and macrophages) and different tissues (e.g., heart, muscle, skin, and pancreas) have been analyzed from this animal, which all produce sufficient reporter gene expression for assessing delivery to specific tissues (Cao et al. (2005) Transplantation 80, 134-9). This animal has also been sectioned to see expression from every tissue examined. When luciferin is delivered iv without a polymer coating, signals can arise from all tissues.

Particle Formation.

A Supercritical Antisolvent System (SAS50, Thar Technologies), was used for all experiments. This instrument is specifically designed for nanoparticle formation on a laboratory scale. It allows mg to gram quantities of particles to be run at a time. In the SAS process, shown in FIG. 1, the compound of interest, the solute, is solubilized in a cosolvent. This cosolvent can contain only the target drug compound or also the polymer being used for encapsulation. This solution is then sprayed, at a set flow rate, into the particle vessel, now filled with a continuous flow of supercritical carbon dioxide. As the cosolvent is expanded into the particle vessel, the cosolvent is immediately solubilized in the supercritical fluid, and the solute will instantly precipitate out of solution. The target compound must therefore be insoluble at the process conditions used, and the cosolvent must be completely soluble. This requires use of the phase diagram of the selected solvents, in order to select the optimal process parameters to be used for a specific compound.

The particles are collected on a filter at the bottom on the particle chamber, and the supercritical fluid (now a mixture of carbon dioxide and cosolvent) are further expanded into a coalescer at a lower pressure. This causes the cosolvent to drop out of solution for further collection, and the carbon dioxide is now a gas which can be recycled and reused.

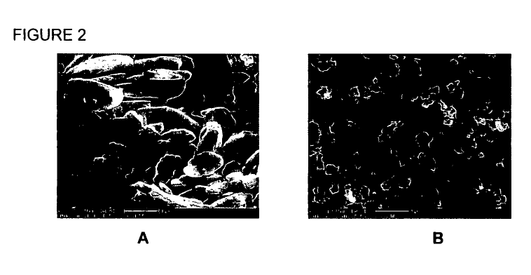

Methanol was chosen as cosolvent for Luciferin, based on previous solubility studies. A range of process parameters were investigated and have allowed successful formation of luciferin particles down to the 100 nm scale. Shown in FIG. 2 are SEM images of luciferin before (2A) and after (2B) our SCF processing technique. A significant reduction in particle size can be observed as well as a uniform size distribution. These preliminary results indicate that the size can be even further reduced by optimization of process conditions.

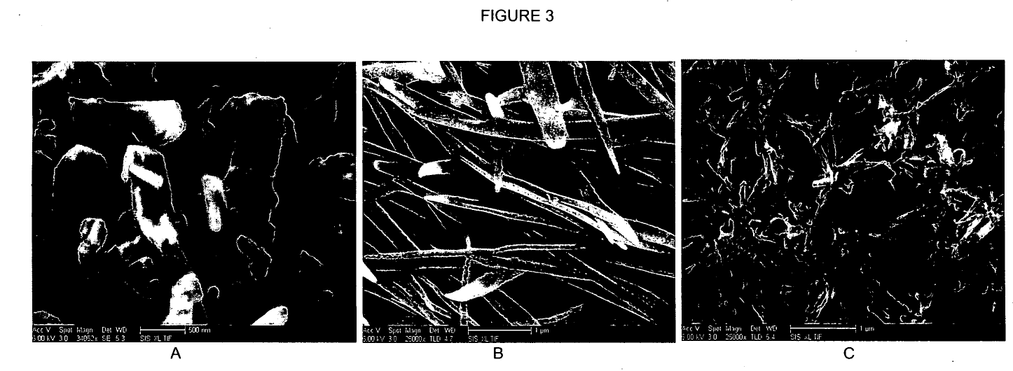

Quercetin. Solubility studies for Quercetin have been performed in a range of cosolvents as well as process conditions. FIG. 3 shows the significant effect the choice of cosolvent can have. The SEM (A) on the left shows quercetin as received, before SAS processing. With all other process parameters being the same, the particle size is changed when switching from methanol (B) to isopropanol (C) as cosolvents, which may be caused by the different solubilities of the two cosolvents at the specific process conditions selected.

The antioxidative activity of quercetin has not been diminished by this supercritical fluid process for nanoparticle formation. A radical scavenger DPPH (2,2,-diphenyl-1-picrylhydrazyl) was used to measure the EC₅₀ value of DPPH at maximum UV absorbance before and after addition of quercetin. The EC₅₀ value was the same before and after SAS processing, showing no loss in antioxidative activity during the process.

Particle Encapsulation

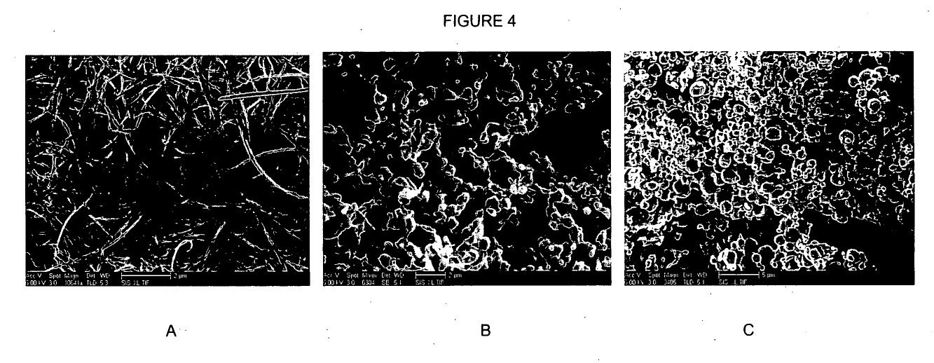

Quercetin. Quercetin has been encapsulated with PLA (MW 50,000 and 100,000) using the following process parameters: T=40 C, P=100 bar, CO2 flow rate 50 g/min, 5:100 quercetin:polymer ratio using methanol as the cosolvent. Results can be seen in FIGS. 4A-4C. Moreover, we have demonstrated that the antioxidative activity of quercetin has not been diminished by this supercritical fluid process for nanoparticle formation. A radical scavenger DPPH (2,2,-diphenyl-1-picrylhydrazyl) was used to measure the EC₅₀ value of DPPH at maximum UV absorbance before and after addition of quercetin. The EC₅₀ value was the same before and after SAS processing, showing no loss in antioxidative activity during the process.

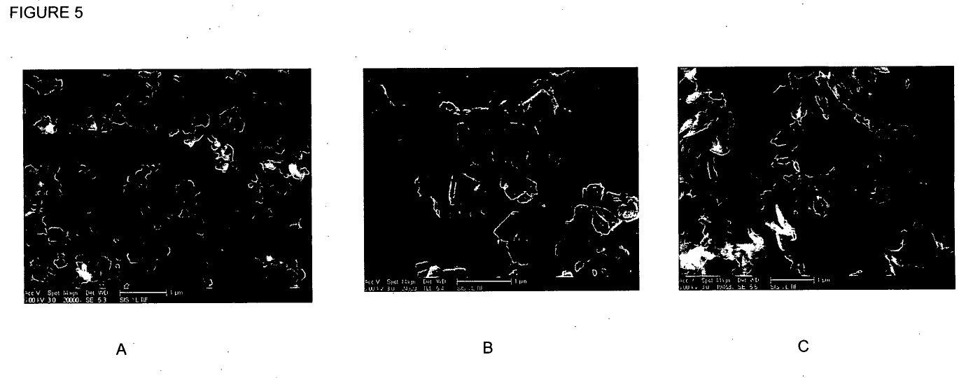

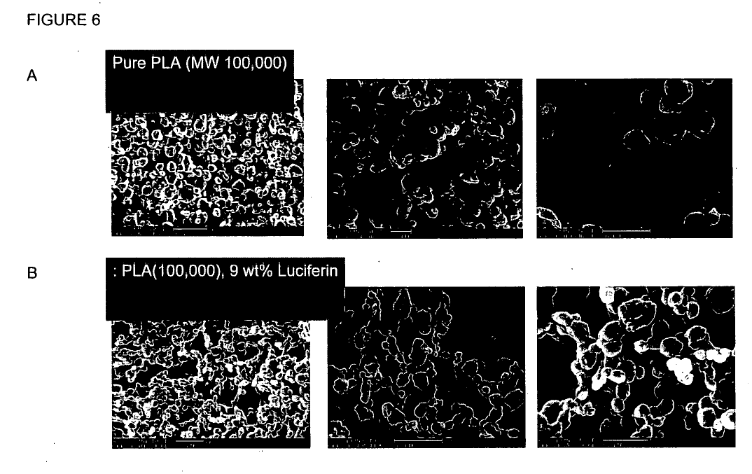

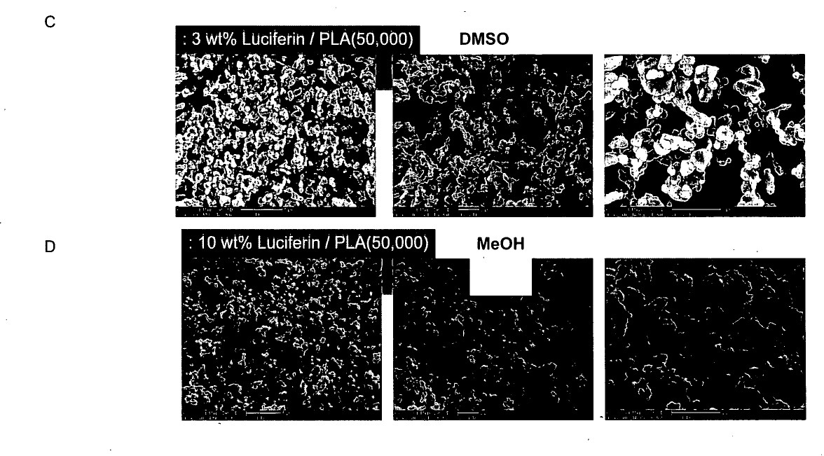

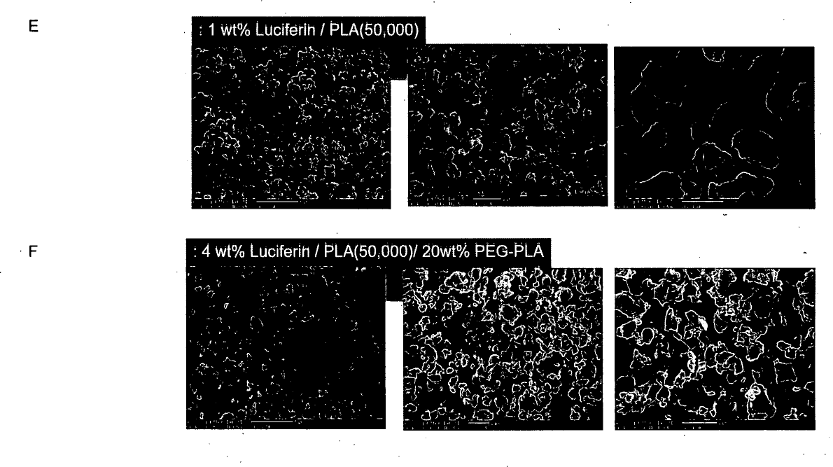

Luciferin. Encapsulation has been performed using luciferin and several different polymers; PLGA, PLA, chitosan, and PEG-PLA. Using PLGA, and chitosan, the same process conditions were used as for luciferin in FIG. 2. PLGA was dissolved in ethyl acetate as a cosolvent and chitosan was dissolved in 0.1 M acetic acid. For both experiments the polymer solutions were mixed with the luciferin/methanol solution and sprayed simultaneously into the particle vessel. These initial results show as expected an increase in particle size and change in morphology, as compared to luciferin alone (FIG. 5).

Using PLA the cosolvent was switched to dichloromethane/methanol mixture and the T=40° C., P=100 bar, CO₂ flow rate 50 g/min, and injection flow rate=1 mL/min. The ratio of luciferin to polymer as well as the encapsulation ratio after the SAS process was also measured using fluorescence and is summarized in Table 1 for a selection of typical runs.

TABLE 1
Results of encapsulation experiments using luciferin
and a mixture of polymer mixtures.

| | | | | wt % | % luci- |
|---|---|---|---|---|---|
| | | | Initial | luciferin | ferin |
| | | | wt % | after | encap- |
| | Polymer | Cosolvents | luc. | SAS | sulated |
| A | PLA(100,000) | CH2Cl2/DMSO | 9.1 | 1.3 | 77 |
| B | PLA(50,000 | CH2Cl2/DMSO | 9.1 | 3.4 | 80 |
| C | PLA(50,000) | CH2Cl2/DMSO | 1.0 | 1.2 | 75 |
| D | PLA(100,000) | CH2Cl2/DMSO | 1.0 | 0.8 | 82 |
| E | PLA(50,000) | CH2Cl2/MeOH | 9.0 | 10.9 | 58 |
| F | PLA(50,000) | CH2Cl2/MeOH | 1.0 | 1.1 | 37 |
| G | PLA(50,000)/20 wt | CH2Cl2/MeOH | 4.5 | 2.9 | 55 |
| | % PEG(5,000)- | | | | |
| | PLA(5,000) | | | | |
| H | PLA(50,000)/20 wt | CH2Cl2/MeOH | 4.2 | 5.3 | 64 |
| | % Pluronic F68 | | | | |
| I | PLA(50,000)/5 wt | CH2Cl2/MeOH | 5.1 | 6.3 | 62 |
| | % Pluronic F68 | | | | |

SEM images of the particles in Table 1 are shown below:

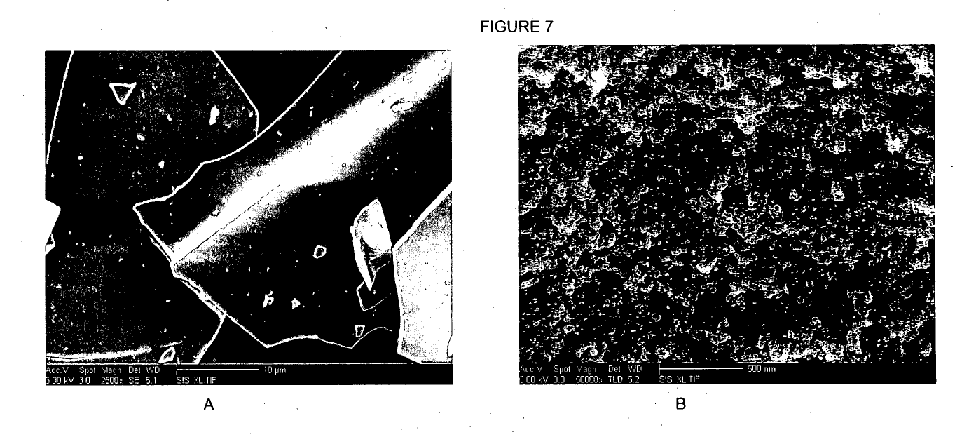

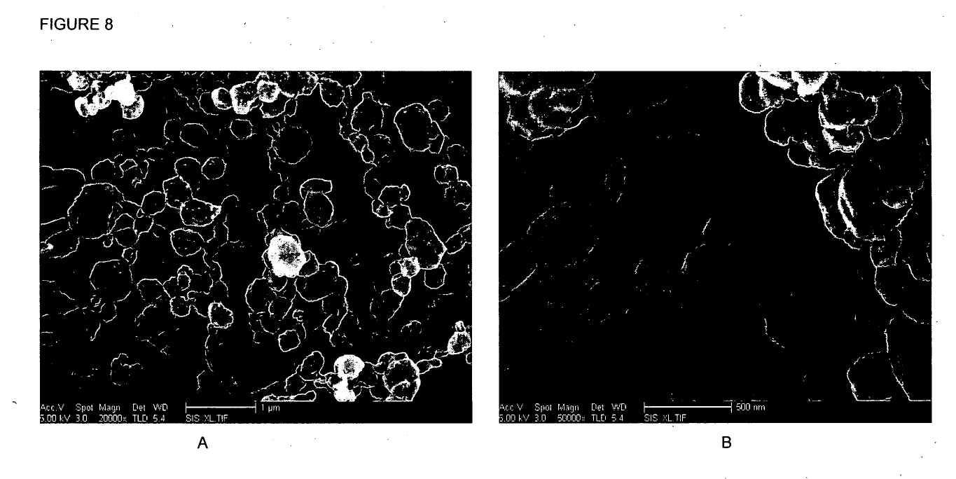

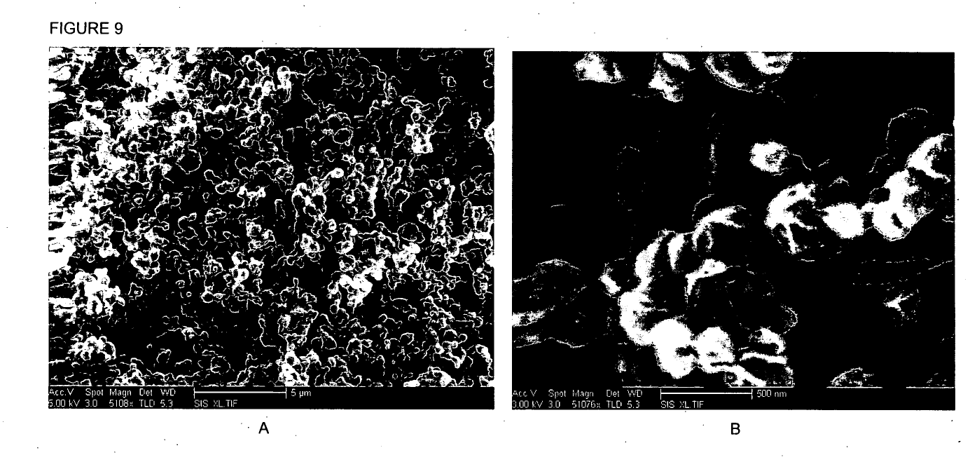

tRNA. As a model for nucleic acid encapsulation, the encapsulation and nanoparticle formation process was optimized for tRNA. tRNA nanoparticles were formed by dissolving 5 mg tRNA in 100 μL H2O and adding 10 mL methanol. The SAS conditions were T=40 C, P=100 bar, CO₂ flow rate=150 g/min and injection flow rate 1 mL/min. SEM images of tRNA before and after SAS (FIG. 7) shows a significant decrease in particle size after SAS. tRNA was also encapsulated with PLA using either 50,000 or 100,000 MW PLA, see FIGS. 8 and 9.

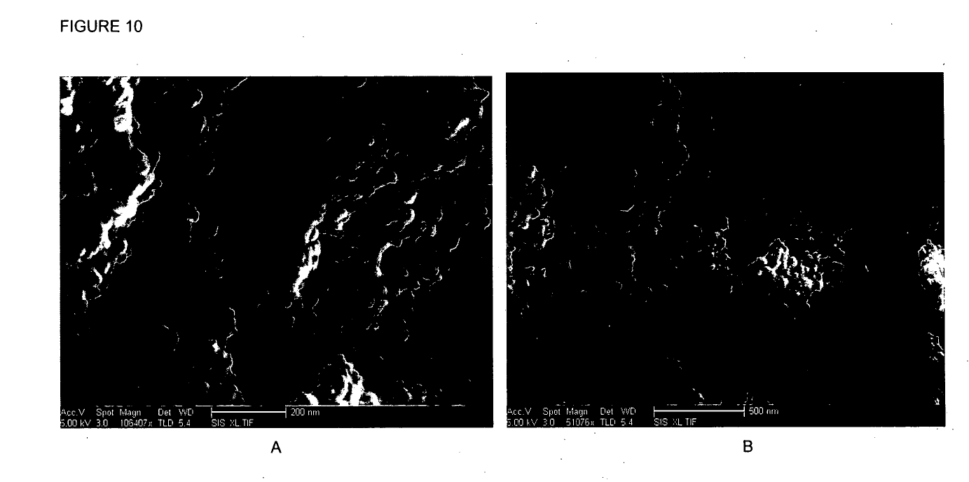

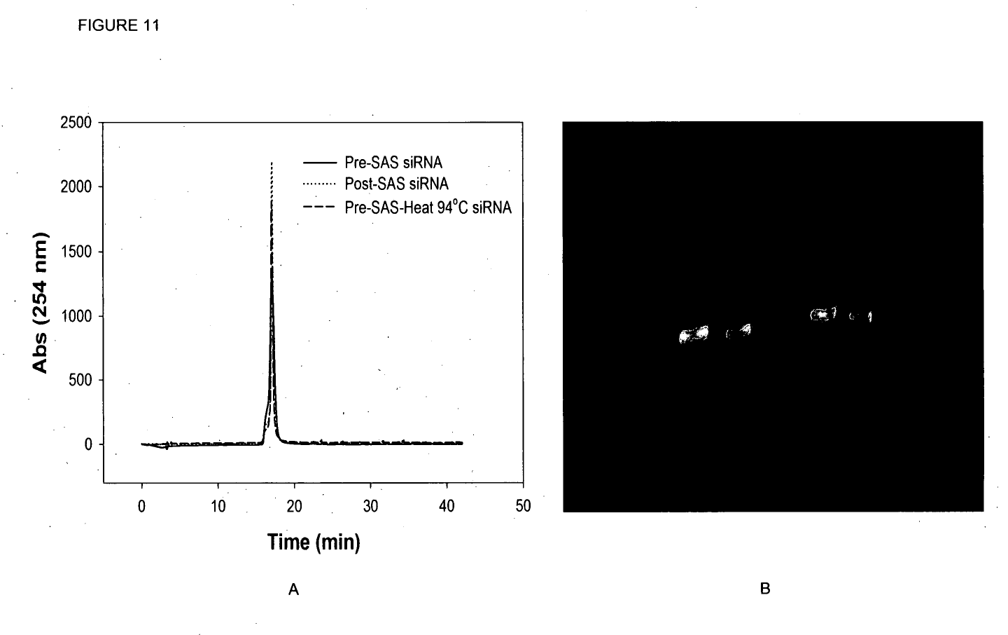

siRNA. K6a N171K.12 siRNA was dissolved at 100 mg/mL in 0.1×PBS. 100 μL solution containing 10 mg siRNA was diluted to 10 mL with MeOH. SAS was performed at 80 C, 90 bar, 1 ml/min injection, 50 g/min CO₂ Collected 3.918 mg of white, fluffy particles. Transferred 1.034 mg of collected particles for testing. SEM analysis of siRNA after the SAS process are shown in FIG. 10. The siRNA was also analyzed by high pressure liquid chromatography (HLPC) and polyacrylamide gel electrophoresis (PAGE) with both showed no evidence of degradation due to the SAS process, FIG. 11.

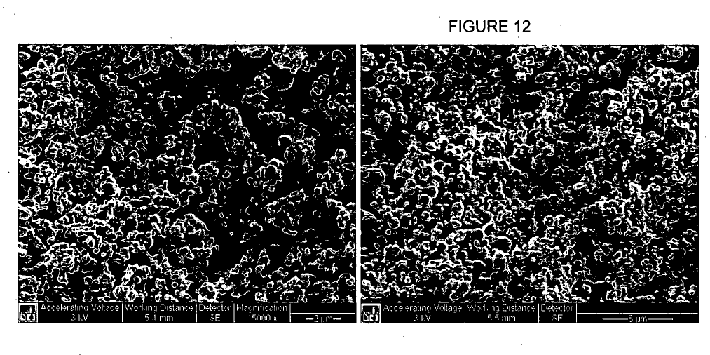

siRNA was encapsulated according to the following protocol. 2 mg K6a N171K.12 siRNA was dissolved at 100 μL H₂O and diluted to 10 mL with MeOH. 40 mg PLA (MW 100,000) was dissolved in 20 mL dichloromethane and added to the siRNA solution. SAS was performed at 40° C., 100 bar, 1 ml/min injection, 150 g/min CO₂. SEM analysis of encapsulated siRNA after the SAS process are shown in FIG. 12.

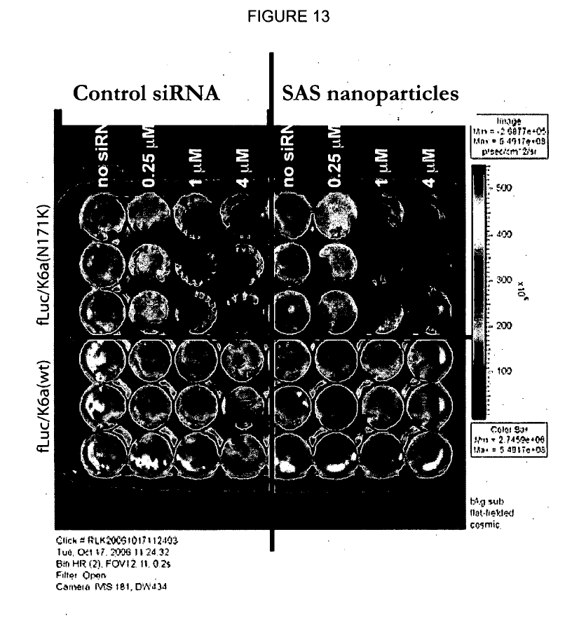

An in vitro functional assay was performed to ensure remained biological activity of the siRNA after SAS. The K6aN171K.12 siRNA targets the K6a keratin-producing gene (PC). This K6a gene is linked to a luciferase gene, so their expression is linked. The keratin gene expression is then determined by light emitted, which means that as bioluminescence decreases the amount of keratin production is also decreasing, which is a sign of the siRNA particles silencing the expression. This silencing was tested with siRNA before and after SAS, as shown in FIG. 13. The data shows that there is no difference in activity before and after SAS process.

In vivo bioluminescence imaging (BLI) as a measure of tumor burden for preclinical efficacy studies. BLI utilizes reporter genes that encode one of any number of light-generating enzymes to tag a specific biological process. The family of luciferase enzymes present in certain bacteria, marine crustaceans, fish, and insects, consists of proteins that can generate visible light through the oxidation of an enzyme-specific substrate in the presence of oxygen and, usually, a source of energy, i.e., ATP. Part of the chemical energy during these reactions is subsequently released as visible light. A significant advantage of luciferases as optical indicators in live mammalian cells and tissues is the inherently low background, given the near absence of endogenous light from these cells. Low levels of light generated within a living animal can escape the absorbing and scattering environment of mammalian tissues and be detected externally; this comprises the method of BLI and has lead to luciferase becoming the reporter of choice for many in vivo applications. The method is extremely sensitive, capable of detecting 100-1000 PC3M cells through the tissues of living animals. Moreover, the relationship between cell number and signal intensity is linear provided that the dynamics of the system do not change (i.e., cell movement, necrotic tissue, and etc.), thus providing a readily accessible measure of tumor burden and response to therapy. Spectral imaging can improve signal quantification.

The most commonly used luciferase for both in vitro and in vivo applications is the luc gene obtained from the North American firefly, Photinus Pyralis, which encodes a 550 amino acid protein. The native substrate for this reaction, D-luciferin (D-(−)-2-(6′-hydroxy-2′-benzothiazolyl) thiazoline-4-carboxylic acid), is converted into oxyluciferin in a Mg²⁺ and ATP-dependent process. Use of this luciferase reaction in vivo, as a marker of gene expression, cell growth, or enzymatic activity requires that the substrate be non-toxic and that when added exogenously (via intraperitoneal, i.p. or intravenous injection, or via inhalation) be well distributed in the body such that the substrate is not limiting. The compound, luciferin, has a number of properties that make it well suited as a substrate for in vivo imaging. From the use of luciferin as a substrate for BLI and studies of biodistribution by tissue sampling and imaging it has been found that this compound can cross cell membranes, biological barriers such as the blood-brain and placental barriers, and has a relatively long circulation time in the body. These properties have prompted investigation of this molecule and its derivatives, as a novel clinical imaging agent and to improve its properties for in vivo imaging in preclinical studies. The biodistribution of the particles can be investigated using transgenic reporter mice where the transgene is comprised of a constitutive ubiquitous promoter (hybrid of CMV enhancer and chicken β-actin prometer) upstream of the bicistronic gene encoding luciferase and green fluorescent protein (GFP). This reporter mouse will serve as an indicator of where the luciferin nanoparticles enter into cells and tissues in the body.

A class of molecules based on luciferin, the substrate for firefly luciferase, have been investigated. By replacing the hydroxyl group on the molecule with an amino group (amino-luciferin), the compound becomes a pseudo amino acid that can be incorporated into peptides and used as a sensor for transport) and a novel imaging tool. Use of luciferyl peptides will enable rapid analyses of the delivery of peptides in vivo and we will use the strengths of this class of compounds to advance the testing and delivery of peptides to locate specific cells with unique targets such as proteases. The compatibility of SAS with peptides enables the development of novel imaging agents based on the techniques described herein. A target for this approach is prostate specific antigen (PSA) which is over expressed in the tumor region in prostate cancer. This is an extracellular protease that is known to be in an active form, and previous attempts to target the tumor using prodrug approach have not been successful.

PSA is a chymotrypsin-like enzyme which specifically cleaves the C-terminus of glutamine (Q) in the peptide sequence, SKLQ-aminoluciferin. Prodrugs have been used to take advantage of this specificity of PSA. Using the L2G85 mice, it was shown that SKLQ-aminoluciferin is distributed non-specifically throughout the animal, thus reducing the tumor-targeting potential of the peptide which is a model prodrug. The peptide was also found to be unstable in the body and was acted upon by various enzymes. Where the peptides or prodrugs are encapsulated with polymer, the current shortcomings in delivery of tumor-specific peptides is overcome. The peptides or drugs can be encapsulated with a protective polymer coating, and the payload released in the vascularized tumor regions. This approach prevents non-specific distribution of the drug and ensures stability of the peptide/drug, and utilizes the active proteases that are specific to a disease state to activate a prodrug at the region of interest.

The biodistribution of luciferin and other luciferyl-derivatives has been investigated in using BLI with L2G85 as the platform for detection. The strategies developed for these studies are utilized in the study of biodistribution of luciferin nanoparticles. The nanoparticles are also tested using a xenograft model of prostate cancer. Appropriate cell lines have been developed for conducting experiments to study human prostate cancer models in animals. LNCaP is a PSA+ cell line that is a widely used model for prostate cancer. Luciferase was introduced into the cells using pcDNA-fLuc plasmid vector and Lipofectamine 2000D from Invitrogen. Ten of the brightest clones were isolated from the luc+ LNCaP cells and are used as targets for luficerin delivery. PC3M cells are PSA negative, and are a useful tool for tumor targeting studies since the cells grow quickly in immune deficient mice and have been extensively studied. Both LNCaP and PC3M cells have successfully been implanted into SCID mice, and their growth kinetics have been successfully quantified using BLI. Luciferase present in the cells is used as the read out for delivery of luciferin and can also be used as for assessing tumor burden when we transition to testing chemotherapeutic agents. The delivery of the nanoparticles is investigated against these prostate cancer xenograft models.

Luciferin is a substrate for luciferase producing a bioluminescent signal of 560 nm at room temperature, and is also a fluorophore with Eₓ at 330 nm and Eₘ at 520 nm. It also absorbs energy at 224 nm. The release of luciferin from the nanoparticles in various buffers, such as PBS and cell culture media such as RPMI or HBSS without phenol-red indicator can be quantified using the absorbance at 224 nm. For instance, known concentrations of the nanoparticles can be dissolved in appropriate media with stirring. Aliquots of the media can then be removed at requisite time intervals and their absorbance spectra recorded at 224 nm. The concentration of luciferin in the media can be assessed by comparison to a standard curve.

Several nanoparticles of luciferin encapsulated with PLGA of different copolymer ratios were tested using this method to show the time-release if luciferin.

Upon release from the nanoparticles, the luciferin diffuses across the cellular membrane and will be processed by luciferase resulting in the emission of light. This provides a functional readout where the integrity of the released compound is assessed in living cells and also in living animals. In cell culture assays the treatment of the data yields kinetic parameters that correlate well with the partitioning coefficient (P), which provide an estimate of the bioavailability of the luciferin. These results are extended to animal studies and the data correlated to the biodistribution of luciferin in target tissues.

Animal studies are performed using transgenic L2G85 mice that have been engineered to ubiquitously express luciferase throughout the body with a β-actin promoter. The mice are administered the luciferin nanoparticles in PBS intravenously. Since the mice express luciferase in all tissues that have been analyzed at significant levels for in vivo detection, very low amounts of luciferin are required to elicit a bioluminescence response. This provides a wide dynamic range for assessing the concentration luciferin after injection of nanoparticles into mice. The nanoparticles will be localized to specific regions in the animal and following release luciferin will produce a signal that can be assessed at various time intervals to determine kinetics of delivery to target tissues. The BLI from the mouse increases with time, directly proportional to the amount of luciferin released. The emission profile will be markedly different from the profile obtained with luciferin and provides information on the kinetics of luciferin release that is influenced by the composition of the polymer coating.

What is claimed is:

1. A method of generating polymer-encapsulated nanoparticles of a biologically active agent, the method comprising:
solubilizing the active agent and a polymer in a cosolvent to provide a mixture;
spraying the mixture at a set flow rate into a vessel filled with a continuous flow of super-critical CO₂ under process conditions wherein the active agent is insoluble, and the cosolvent is completely soluble;
wherein the active agent is precipitated in encapsulated, nanosized particles.

2. The method of claim 1, wherein the nanosized particles are from 10 nm to 10 μm in diameter.

3. The method of claim 1, wherein the active agent is a polynucleotide.

4. The method of claim 1, wherein the polynucleotide is RNA.

5. The method of claim 1, wherein the polynucleotide is DNA.

6. The method of claim 1, wherein the active agent is a polypeptide.

7. The method of claim 1, wherein the active agent is a drug.

8. The method of claim 1, wherein the polymer is a biodegradable polymer.

9. The method of claim 1, wherein the cosolvent is a homogeneous mixture of a first solvent and a second solvent miscible with the first solvent.

10. The method of claim 9, wherein solubilizing comprises the steps of:
solubilizing the polymer in the first solvent;
solubilizing the active agent in the second solvent;
mixing the first solvent and second solvent to provide a homogeneous mixture.

11. The method of claim 1, wherein the active agent is solubilized at a concentration from about 0.001 mg/ml to about 10 mg/ml.

12. The method of claim 11, where the ratio of compound to polymer as a weight percentage is from about 1:1000 to about 1:5.

13. The method of claim 1, wherein supercritical CO₂ flow rate is at least about 1 g/min of CO₂ and not more than about 1000 g/min of CO₂.

14. The method of claim 1, where the CO₂ is at a subcritical temperature.

15. The method of claim 13, wherein the cosolvent flow rate is at least about 0.01 ml/minute and not more than about 100 ml/minute.

16. The method of claim 1, wherein temperature in the process condition is below the glass transition temperature of the polymer.

17. The method of claim 16, wherein the temperature is from 20° C. to 80° C.

18. The method of claim 17, wherein the temperature is from about 40° C. to about 45° C.

19. The method of claim 1, where pressure in the process condition is at least about 50 bar and not more than about 1000 bar.

20. A population of polymer-encapsulated nanoparticles of a biologically active agent produced by the method according to claim 1.

21. The population of polymer-encapsulated nanoparticles of claim 20, further comprising a pharmaceutically acceptable excipient.

22. The population of polymer-encapsulated nanoparticles of claim 19, wherein the nanoparticles comprise a solid core that is substantially pure biologically active agent.

23. The population of claim 22, wherein the population has a substantially homogeneous size.

24. A population of nanoparticles of a polynucleotide, wherein the nanoparticles are from 10 nm to 100 nm in diameter.

25. The population of nanoparticles according to claim 24, wherein the nanoparticles comprise a polymer coating.

26. The population of nanoparticles according to claim 25, wherein the polynucleotide is RNA.

27. The population of nanoparticles according to claim 26, wherein the RNA is an RNAi molecule.

28. The population of nanoparticles of claim 24, further comprising a pharmaceutically acceptable excipient.

29. The population of nanoparticles of claim 27, wherein the RNAi has been treated to increase hydrophobicity prior to encapsulation.
